(19) United States
(12) Patent Application Publication
(10) Pub. No.: US 2006/0079750 A1
(43) Pub. Date: Apr. 13, 2006

(54) Systems and methods for localizing vascular architecture, and evaluation and monitoring of functional behavior of same

(76) Inventors: Mark A. Fauci, Patchogue, NY (US); Matthew A. Salvitti, Patchogue, NY (US); Marek Pawlowski, Glen Cove, NY (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/170,364

(22) Filed: Jun. 29, 2005

Related U.S. Application Data
(60) Provisional application No. 60/585,806, filed on Jul. 6, 2004.

(51) Int. Cl. A61B 5/05

(52) U.S. Cl. 600/407

(57) ABSTRACT

A system is provided utilizing dynamic imaging protocol to localize vascular architecture, and to evaluate and monitor functional behavior of the vascular architecture for pre-operative, post-operative and diagnostic purposes. The system includes, among other things, a scanner having within its objective portion an assembly for capturing a photon beam emitted from an object being monitored. The system is also provided with a detection network designed to convert, into electronic signals, data correlated from the beam. A processor can be provided for generating discrete image data from the electronic signals for subsequent display as an image. The system can also include a display for viewing the image data. The system can further include a ruler for positioning on the object being monitored to permit subsequent translation of the image viewed in the display onto the object. Methods for evaluating, monitoring, and localizing the vascular architecture are also provided.

### RELATED U.S. APPLICATIONS

The present application claims priority to U.S. application Ser. No. 60/585,806, filed Jul. 6, 2004, which application is hereby incorporated herein by reference.

### TECHNICAL FIELD

The present invention relates to a system and method using dynamic infrared imaging for localizing vascular architecture, and for evaluating and monitoring functional behavior of the vascular architecture for pre-operative, post-operative, and diagnostic purposes.

### BACKGROUND ART

The concept of infrared imaging for biomedical applications has been explored for some time. The early technology used, unfortunately, had neither the sensitivity, resolution nor speed to be of substantial value. Infrared imaging has now advanced to where it is being used for a range of applications in medicine, and has multiple advantages over conventional medical imaging techniques, including, low cost, no ionizing radiation and minimal need for contrasting agents. The existing infrared systems, however, are limited in sensitivity and speed. As such, the use of these systems for identifying, for instance, vessel architecture, can be crude and can require the use of contrast enhancement techniques, such as cooling the area of interest or the use of contrast agents.

Moreover, existing medical imaging systems are limited to collecting information about the tissue physiology in a single band (i.e., wavelength spectrum) of emission. In addition, since these systems typically use static measurements of spatial distribution of photon flux, they may display only information pertaining to, for example, the infrared flux at a particular single band, rather than information in multiple infrared bands or dynamics of infrared photon flux over time. Due to the nature of infrared energy, namely the absorption of specific bands of infrared photons by certain components of biological tissue, such as gasses and fluids, there would be significant advantages in employing a multiband detector that could analyze and display multiple bands of infrared energy simultaneously. A properly designed system would permit the direct analysis of gas, fluid and other diagnostically important tissue characteristics.

With certain surgical procedures, such as reconstructive surgery where free tissue transfer may be involved, there can arise problems that prevent a successful outcome of such a procedure. Specifically, the ability to precisely identify the vascular pedicle, the ability to identify the perforator vessels that perfuse the selected free tissue flap, as well as the performance of a vascular anastomosis with the recipient vessels, can determine whether there will be a successful outcome.

Currently, there are commercially available systems that permit the objective localization of the vascular architecture, such as the perforator vessels. These systems, however, do not typically employ infrared imaging. Rather, one approach is to use, for instance, ultrasound Flow Doppler Meter. However, such an approach can be time consuming and may not yield accurate results. Moreover, the use of thermal imaging to localize cutaneous perforators has been discussed in the literature. However, such an approach focuses on temperature variations and requires the application of a cold stimulus (fan cooling, ice packs, etc.) prior to imaging, which can be uncomfortable to a patient, or a contrasting agent, which can generate adverse effects in a patient when delivered within the patient's body and/or when a high level of energy is used. Furthermore, during a pre-operative period, currently available systems may not be able to allow a healthcare provider to accurately evaluate the vascular architecture. Similarly, during a pre-operative or post-operative period, these systems may not allow the provider to essentially instantly evaluate blood perfusion activity in the area of interest, so as to avoid the potential damage or loss of existing or transplanted tissue.

Accordingly, it would be advantageous to provide a system and method that can provide a fast, reliable, accurate, non-invasive approach to the objective localization of vascular architecture to facilitate the evaluation and monitoring of functional behavior of the vascular architecture for pre-operative, post-operative and diagnostic purposes.

### SUMMARY OF THE INVENTION

The present invention provides, in one embodiment, a dynamic imaging system having a scanner designed to include a body portion and an objective portion. The system also includes an assembly, positioned within the objective portion, for splitting a photon beam emitted from an object being monitored into multiple incident rays of different wavelength spectra. The system also includes a detection network designed to receive the multiple incident rays for converting, into electronic signals, data correlated from the respective incident rays. The detection network, in an embodiment, may include an infrared detector. Such a detector may be a quantum well infrared photodetector (QWIP). The system may further include a processor for generating discrete image data from the electronic signals of each respective incident ray. The image data regarding the object being monitored may subsequently be viewed on a display. The system can also include at least one ruler for positioning on the object being monitored to permit subsequent translation of the image viewed in the display onto the object.

The present invention provides in another embodiment, a dynamic imaging system having, among other things, an objective portion through which a photon beam emitted from an object being monitored may be directed. The system, in an embodiment, may include a plurality of mirrors within the objective portion for splitting the photon beam into multiple incident rays, each of a different wavelength spectrum. At least one detector may be provided and tuned to a specific wavelength spectrum of the incident ray it is collecting from the corresponding mirror, so as to convert, into electronic signals, data correlated from the incident ray. The system further includes a processor for generating discrete image data from the electronic signals of each respective incident ray. The image data in connection with the object may subsequently be viewed on a display. The system can also include at least one ruler for positioning on the object being monitored to permit subsequent translation of the image viewed in the display onto the object.

In another embodiment, the present invention provides a method for evaluating vascular architecture of a tissue area on a patient. The method includes initially maintaining, at substantially normal body temperature, the tissue area having the vascular architecture of interest on the patient. Next, photon flux emitted from the tissue area may be detected. In one embodiment, a stream of individual frames of data from detected photon flux may be collected. Thereafter, data collected from the detected photon flux may be processed. Subsequently, contrast between the vascular architecture and surrounding tissue within the area being scanned may be enhanced, and an image from the processed data may be displayed for viewing. In accordance with an embodiment of the invention, the image displayed may be used for pre-operative and/or post-operative evaluation.

In further embodiment, a method for localizing perforator vessels is provided. The method includes initially maintaining, at substantially normal body temperature, the tissue area having the vascular architecture of interest on the patient. Next, a reference point may be placed on the tissue area to assist in subsequent localization. Thereafter, the tissue area may be scanned with an infrared camera, so that the reference point is within the field of scan to detect photon flux emitted from the tissue area. In one embodiment, the reference point may be provided by placing a ruler having calibrated markings onto the tissue area. Then, data collected from the detected photon flux may be processed. Subsequently, contrast between the vascular architecture and surrounding tissue within the area being scanned may be enhanced, and an image from the processed data may be displayed for viewing. An electronic illustration of a grid having a coordinate system may next be positioned on the image displaying the perforator vessels, such that the origin of the grid is situated relative to the reference point captured during the scan. Then, the location of the perforator vessels within the grid may be identified. Thereafter, while maintaining orientation of the grid relative to reference point, the location of the perforator vessels within the grid may be translated to the tissue area previously scanned. Subsequently, the location of the perforator vessels as identified within grid may be marked on the tissue area.

### BRIEF DESCRIPTION OF DRAWINGS

FIG. 1 illustrates a perspective view of a dynamic imaging system of the present invention for use in tissue analysis.

FIG. 2 illustrates a perspective view of another dynamic imaging system of the present invention for use in tissue analysis.

FIG. 3 illustrates the various components of a scanner shown in FIG. 2.

FIG. 4 illustrates an alternate embodiment for the detection component of the scanner shown in FIG. 2.

FIG. 5 illustrates an lens system for use in connection with dynamic imaging system of the present invention.

FIG. 6 illustrates an end view of an alternate lens system for use in connection with the dynamic imaging system of the present invention.

FIG. 7 illustrates a longitudinal section view of the lens system in FIG. 6.

FIG. 8 illustrates a detailed view of a mirror in the lens system in FIG. 6.

FIGS. 9A-B illustrate a various embodiments for a light source for use in connection with the dynamic imaging system of the present invention.

FIG. 10 a ruler for use in connection with the dynamic imaging system of the present invention.

### DESCRIPTION OF SPECIFIC EMBODIMENTS

The present invention, in one embodiment, is directed to a system and method for localizing vascular architecture, and for the evaluation and monitoring of the functional behavior of the vascular architecture for pre-operative, post-operative and diagnostic purposes. The system employs a detection network of at least one detector, single or multiple bands, that is capable of collecting photons of various wavelengths for dynamic imaging of a tissue area of interest. The dynamic imaging system of the present invention may also permit a user to view multiple bands of electromagnetic radiation concurrently as individual images or as a merged or superimposed image.

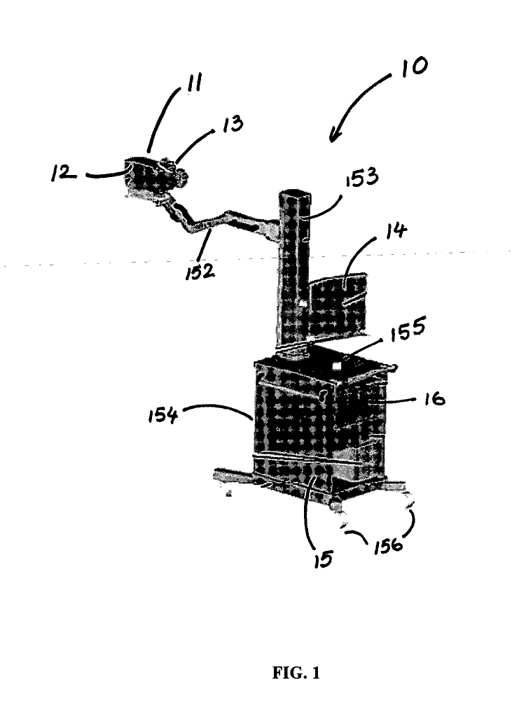

With reference now to FIG. 1, there is illustrated a dynamic imaging system 10 of the present invention. The system 10 includes, in one embodiment, a scanner 11 positioned on a mobile cart 15 for ease of use. The scanner 11 includes a body portion 12, within which detection components of the scanner 11 may be positioned, an objective portion 13 having an optical component for detecting photon flux emitted from a tissue area being analyzed, and a display screen 14 remotely situated on the mobile cart 15. The scanner 11 of system 10, as illustrated, may be similar to that commercially available as the BioScanIR® System from Advanced Biophotonics in Bohemia, N.Y.

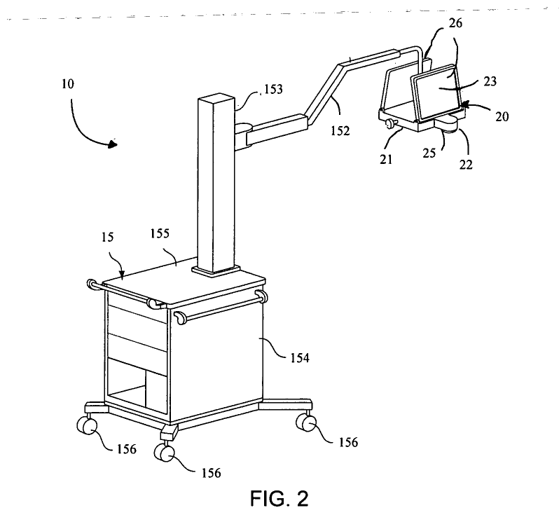

In another embodiment, looking now at FIG. 2, rather than scanner 11, the dynamic imaging system 10 may be equipped with a scanner 20 having a body portion 21 within which detection components may be positioned, an objective portion 22 within which integrated optical components may be housed, and an integrated output display portion 23.

As the body portion 21 and the objective portion 22 are designed to house the working components of the scanner 20, these portions, in one embodiment, may be made from a strong material, such as, a metal, a metal alloy, molded plastic, fiberglass, or a combination thereof. In addition, although illustrated in FIG. 2 with particular designs, it should be appreciated that the body and objective portions, 21 and 22, may be provided with any geometric shape, so long as these portions can accommodate the components for which they have been designed to house. The objective portion 22, in one embodiment, may be designed to be positioned over a target or object 24, e.g. tissue to be analyzed or monitored. The objective portion 22 may include an opening 25 through which a photon beam 26 emitted from object 24 may be directed into the objective portion 22.

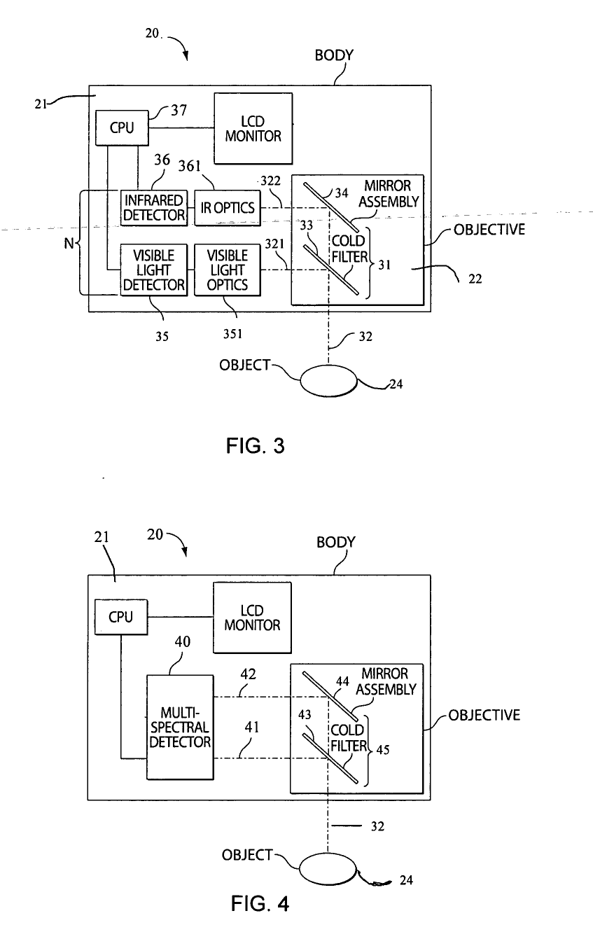

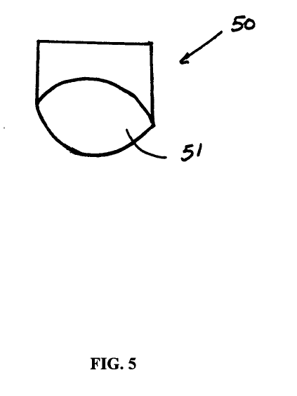

Referring now to FIG. 3, the scanner 20 may include within the objective portion 22 an assembly 31 for separating or splitting photon beam 32 into multiple incident rays, each within a different wavelength spectrum. As illustrated in FIG. 3, assembly 31 may be designed to separate photon beam 32 into at least two incident rays 321 and 322 of different wavelengths. To accomplish this, assembly 31 may be provided, in one embodiment, with an array of at least two mirrors 33 and 34. In accordance with one embodiment of the present invention, mirror 33 may be designed to reflect an incident ray 321 having photons within a specific wavelength spectrum, for instance, visible light spectrum, while being transparent to photons within different wavelength spectra, for instance, near-, mid-, and far-infrared. Mirror 33, accordingly, may be made from any material, for instance, germanium, that may be reflective of photons within the visible spectrum, while being transparent to photons within the infrared spectrum. Mirror 34, on the other hand, may be designed to reflect an incident ray 322 having photons within a specific infrared spectrum, e.g., mid-infrared (8-10 μm). In one embodiment, mirror 34 may be made from any material, for example, glass, stainless steel, chromium, that may be reflective of photons within the mid-infrared spectrum.

Although only two mirrors are illustrated in FIG. 3, it should be appreciated that additional mirrors may be provided, with each mirror in the array being designed to reflect photons within a specific wavelength spectrum, while being transparent to those in other wavelength spectra. The number of mirrors used in the array and the wavelength spectra at which these mirrors may reflect can be dependent on the tissue characteristics to be monitored and the image or images to be generated. Accordingly, should an optical image not be necessary for the particular application, the mirrors provided may, for example, be reflective only to photons within the various infrared spectra. Moreover, should it be desired, the mirrors in the array may be designed to reflect incident rays having photons within various other wavelength regions of the electromagnetic spectrum, e.g., x-rays, ultraviolet etc., depending on the imaging application.

In an alternate embodiment, assembly 31 may include, instead of an array of mirrors, a plurality of filters, shutters, hot/cold prisms, or a combination thereof, each similarly capable of separating and/or reflecting photons within a specific wavelength spectrum while being transparent to photons within other wavelength spectra. Furthermore, to the extent necessary, these mirrors, filters, shutters, and/or hot/cold prisms may be fixed or made to be adjustable in order to vary the angle of incidence.

Still referring to FIG. 3, the scanner 20 may also be provided with a detection network N. In the embodiment shown in FIG. 3, detection network N includes detectors 35 and 36 positioned within the body portion 21 to collect incident rays 321 and 322 respectively. Since incident rays 321 and 322, reflecting off of mirrors 33 and 34 respectively, are of different wavelength spectra, each of detectors 35 and 36 may be tuned to the specific wavelength spectrum for the incident ray it is collecting. In the embodiment illustrated in FIG. 3, detector 35 may be tuned to collect photons in the visible light spectrum, while detector 36 may be tuned to collect photons in the infrared spectrum, for example, mid-infrared spectrum (i.e., 8-10 μm). Detectors 35 and 36, in one embodiment of the invention, may be single-band (i.e., single-spectrum) detectors that are commercially available. Alternatively, infrared detector 36 may be a multi-band (i.e., multi-spectral) detector, capable of receiving photons within various infrared spectra. In other words, should the specific wavelength spectrum of an incident ray to be collected changes from one application to another, detector 36 may still be capable of collecting such an incident ray, should the incident ray comprises photons within the spectra of wavelengths to which the detector 36 may be tuned. In one embodiment, multi-band detector 36 may be a quantum well infrared photodetector (QWIP), such as those disclosed in U.S. Pat. Nos. 5,539,206, 6,184,538, 6,211,529, and 6,642,537, all of which are hereby incorporated herein by reference.

To collect the respective incident rays reflecting off of mirrors 33 and 34, a substantially clear pathway may be provided between the mirrors and detectors 35 and 36. In particular, an opening (not shown) may be provided at a juncture between the body portion 21 and the objective portion 22 that is sufficiently large to permit the incident rays 321 and 322 to move substantially unobstructively therethrough. In one embodiment of the invention, optics components, such as lens 351 and 361 may each be positioned upstream of detectors 35 and 36 respectively to permit the corresponding incident ray to be focused onto the respective detector. In addition, in accordance with an embodiment of the present invention, detectors 35 and 36 may be situated in such a manner so as to allow incident rays 321 and 322 to arrive at the respective detectors substantially perpendicularly to the surface of the detectors. To accommodate this, mirrors 33 and 34 may be fixed at an appropriate angle relative to the detectors 35 and 36, or may be adjustable to vary the angle of incidence.

In general, detectors 35 and 36 may be designed to correlate functional, physical and/or optical data from incident rays 321 and 322 coming from the same spatial and temporal source, i.e., object 24. It should be noted that since the correlated data came from photon beam 32 for all wavelength frequencies, any distortion that might be derived from the beams transmitted at different angles from object 24 may be minimized. In addition to correlating data, detectors 35 and 36 may be designed to convert the correlated data from the respective incident rays 321 and 322 into electronic signals.

It should be appreciated that although FIG. 3 illustrates a network N of two detectors, additional detectors may be provided within network N depending on the number of incident rays that may be generated from the beam separator assembly 31. Alternatively, the scanner 20 may employ only one detector 40 in network N, as shown in FIG. 4. In such an embodiment, the detector 40 may preferably be a multi-spectral detector, for instance, a QWIP multi-spectral detector as noted above, or a single band detector as that used in scanner 11. The use of a multi-spectral detector 40 may permit multiple incident rays, such as rays 41 and 42, each of which comprises a different wavelength spectrum, to be received by the multi-spectral detector 40 for data correlation regarding the object being monitored. To direct these various incident rays on to detector 40, mirrors 43 and 44 of beam splitting assembly 45 may be made adjustable to alter the angle of incidence. Should it be desired, additional reflectors (not shown) may be positioned between each mirror and the detector 40 to adjust the angle at which each incident ray may be received by the detector 40. In particular, the additional reflectors may redirect the pathway of each incident ray to arrive at the detector 40 substantially perpendicularly.

Looking again at FIG. 3, the scanner 20 of the dynamic imaging system 10 may further include a processor 37. Processor 37, in one embodiment, may be designed to receive, as electronic signals, the correlated data from detectors 35 and 36, and to generate, in real time, discrete physical and/or functional data as well as image data regarding the object from the electronic signals using a variety of processing options and capabilities. Such a processor may be similar to those disclosed in U.S. Pat. Nos. 5,810,010, 5,961,466, and 5,999,843, all of which are hereby incorporated herein by reference. For example, processor 37 may be provided with a variety of algorithms so that it may generate, from the infrared data, (i) real time information relating to, for example, blood perfusion, tissue characteristics, minute temperature changes, presence of tumorous growth or abnormal tissue behavior, as well as (ii) functional or physiological image signals of such information in connection with the object or target being monitored and/or observed. Data from the visible spectrum, on the other hand, may be used to generate optical image signals of the object or target being observed. The functional/physiological image signals and the optical image signals may be manipulated by processor 37, through a variety of user input, for subsequent display as either a discrete functional image (i.e., from the infrared spectrum) and a discrete optical image (i.e., from the visible spectrum), or as an integrated multi-spectral image of the object being monitored and observed. The integrated multi-spectral image, in accordance with an embodiment, may be a superimposition of a functional image onto an optical image. In this manner, the integrated image can allow a tending physician to visualize, for example, the functional and physical behavior and/or characteristics within the object (e.g., tissue or organ) being monitored and observed.

The processor 37 may also be designed to implement various additional applications, for instance, (a) Pattern Recognition, (b) Dimensional Calibration Application, to assist in the calibration of an image that is being captured, (c) Grid Application, in which a grid with coordinates may be overlaid on the captured image to permit manipulation or modification of the image for subsequent activities, and (d) Measurement Application, for measuring the distances of the image for subsequent transfer of the tissue onto the body of the patient.

In one embodiment of the present invention, processor 37 may be positioned internally within the body portion 21 of the scanner 20, as illustrated in FIG. 3.

Alternatively, processor 37 may be positioned externally of the body portion 21 and remotely from scanner 20. Although described in connection with the scanner 20, processor 37 can be used in connection with scanner 11. Scanner 11, as illustrated in FIG. 1, includes an external processor 16 that is remotely positioned on cart 15 away from the body 12. Whether the processor 37 is within or outside of the body portion 21, electronic signals from the detectors 35 and 36 may be transmitted to the processor 37, in an embodiment, via wires. In an alternate embodiment, where the processor 37 is positioned remotely from the scanner, electronic signals from the detectors 35 and 36 may be transmitted wirelessly to processor 37.

To permit visualization of the image signals generated by the processor 37, the scanner of the present invention may be provided with a display system, such as screen 14 on cart 15 in FIG. 1, or screens 26 located at the output display portion 23 of the scanner 20 in FIG. 2. In the embodiment shown in FIG. 2, screens 26 may be positioned atop the body portion 21 and pivotally connected thereto. The pivotal connection of the screens 26 to the body portion 21 allows the screens 26 to be moved into a substantially upright position, as shown in FIG. 2, for viewing, or folded substantially flush against the body portion 21 when not in use. Should it be desired, screens 26 may also be designed to rotate circumferentially atop the body portion 21, so that a user may avoid having to relocate his/her position when relocation may be difficult. Screens 26 may also be provided remotely (not shown) from the body portion 21. In such an embodiment, the remotely available screens 26 may be used in substitution or in addition to the screens on the body portion 21. It should be appreciated that the display system of the present invention may include two or more screens to permit multiple users, for example, a tending physician and an assistant, to comfortably view the images being displayed thereon. However, it can be well envisioned that only one screen may be provided. Screen 14 (FIG. 1) or screens 26 (FIG. 2), in one embodiment, may be commercially available LCD screens, or any other display device capable displaying images for viewing by the user.

Looking now to 5, in one embodiment of the present invention, the dynamic imaging system 10 may be equipped with lens system 50 for generating, among other things, infrared images of the object being monitored and observed. The lens system 50 may include a an infrared lens 51 for positioning over an object being monitored to collect photon flux emitted therefrom. This single lens system 50 may be used in connection with scanner 11 or scanner 20 of the dynamic imaging system 10.

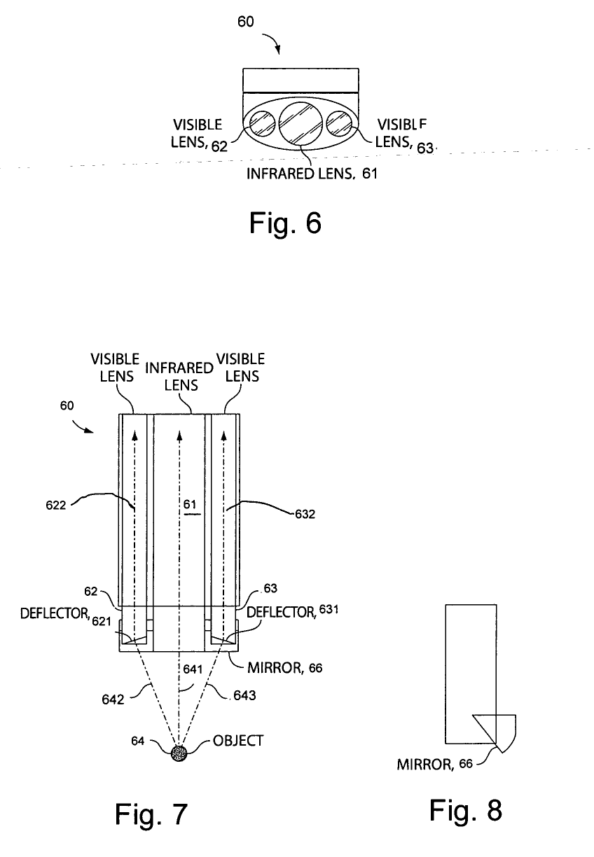

In an alternate embodiment, looking now at FIGS. 6-8, the system 10 may be provided with lens system 60 for generating, among other things, binocular or three dimensional (3-D) images of the object being monitored and observed. Lens system 60, in an embodiment, may be coupled to the opening 25 of the objective portion 22, shown in FIG. 2, and may include at least three lenses, for instance, a center lens 61, and side lenses 62 and 63. The center lens 61 and side lenses 62 and 63 may be situated so as to be directed at a same focal point of an object 64 being observed. The center lens 61, in an embodiment, may be an infrared lens, and may be positioned over object 64 to collect photon beam 641 emitted therefrom. Side lenses 62 and 63, in one embodiment, may be visible light lenses, and may be positioned so that each side lens can also be directed at the same focal point to which the center lens 61 may be focused. To permit the side lenses 62 and 63 to be focused at the same focal point as that by the center lens 61, deflectors 621 and 631 may be used to capture photon beams 642 and 643 from the object 64 and redirect these beams in the manner shown by arrows 622 and 632. Deflectors 621 and 631, in on embodiment, may be adjustable to permit the side lenses 62 and 63 to capture photon beams from the same focal point, taking into account the distance at which the side lenses 62 and 63 may be placed from the object 64.

It should be noted that although the lenses 61, 62 and 63 are illustrated as separate confocal lenses, each with the ability to focus on a similar focal point concurrently as the others, these lenses may not necessarily be separate or discrete in design. Instead, they may be configured to be integral with one another. In addition, fewer or more than three lenses may be used. Regardless of the configuration or design, and depending on the imaging application, the lens system 60 may be made to collect photon beams within various other wavelength regions of the electromagnetic spectrum, e.g., x-rays, ultraviolet, etc.

The lens system 60, as illustrated in FIGS. 7 and 8, may also include, in one embodiment, a mirror assembly 66 positioned adjacent to the deflectors 621 and 631. The mirror assembly 66, in an embodiment of the invention, may be hingedly connected to the lens system 60 adjacent to the deflectors 621 and 631, so that the view by lenses 61, 62 and 63 may be adjusted to any angle from the normal incident.

The lens system 60, when positioned over object 64, may collect photon beams through each of the center lens 61 and side lenses 62 and 63. These beams, in accordance with an embodiment, may be directed as incident rays to three separate detectors similar to those detectors in FIG. 3. In particular, each of these detectors may be tuned to the specific wavelength spectrum of the incident ray it is receiving. The electronic signals generated by the detectors from the correlated data from the respective incident rays can then be transmitted to a processor similar to that shown in FIG. 3 for processing into respective functional and optical image signals. The functional and image signals may thereafter be manipulated and displayed either as separate (i.e., single spectrum) or merged (i.e., multi-spectral) 2D and/or 3D images.

In accordance with one embodiment of the present invention, the lens systems 50 and 60 may be employed without the utilization of a beam splitting assembly, for example, assembly 31 in FIG. 3. However, such an assembly may still be used should the photon beam collected through, for instance, the infrared lens, needs to be separated into various specific infrared spectra, e.g., near-, mid-, and/or far-infrared.

The lens systems 50 and 60 may also be equipped with a focus or zoom component. In this manner, a user may be able to, among other things, view functional and optical images of small regions of interest with greater control, including the ability to control the presentation of the field of view, to study the subject tissue at normal and at magnified settings, to obtain a substantially clear and focused image at varying magnification or distance, and to obtain a field of depth which can facilitate eye hand coordination, while performing the surgical procedure. It should be appreciated that the use of multiple discrete lenses in lens system 60 can minimize issues typically associated with image degradation when approaching from a single lens solution.

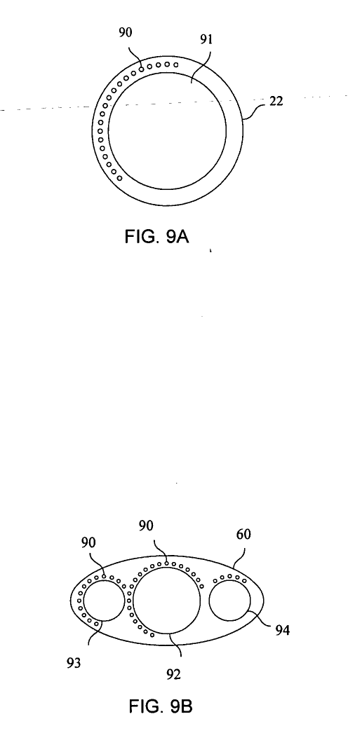

In another embodiment of the present invention, the lens systems 50 and 60 of dynamic imaging system 10 may be equipped with one or more light sources for illuminating the object being monitored and viewed. As illustrated in FIGS. 9A-B, light source 90 may be situated, in one embodiment, about the opening 91 of the objective portion 22. In an embodiment whereby lens system 60 may be used, light source 90 may be situated about center lens 92 and/or side lenses 93 and 94. Light source 90, in accordance with one embodiment, may be a fiber optic light source, or any source which may generate diffuse illumination at the appropriate frequency on to the object being monitored and observed. The presence of light source 90, in one embodiment, can enhance the quality of the image data either by providing additional illumination and/or through spectral analysis, fluorescence or other means.

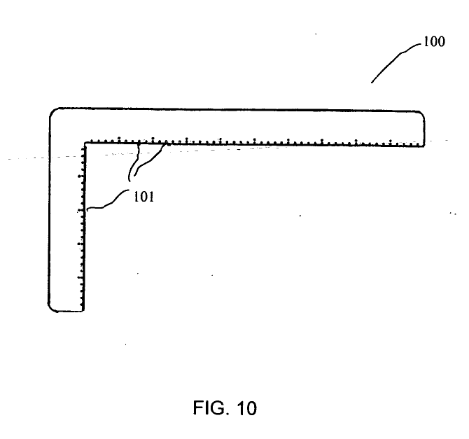

The dynamic imaging system 10 of the present invention further includes, in one embodiment, at least one ruler 100, such as that illustrated in FIG. 10. As shown, ruler 100 may be provided with holes or perforations 101 at predetermined calibrated distances. Multiple rulers of different lengths may be provided for use in connection with the system 10, depending on the dimensions of the tissue area being analyzed and/or monitored. In addition to the ruler 100, the system 10 may also include specially shaped distance gauges (not shown) of calibrated length. The dimensions and shape of the ruler 100 and gauges may be of various sizes and geometric shape, depending on the dimensions of the investigated body part. In one embodiment, the shape of the ruler 100 and gauges may be automatically recognized by a pattern recognition software, while the dimensional calibration of the image may be performed automatically. Moreover, the ruler 100 may be made, in an embodiment, from a material that permits the ruler 100 to be visible in any spectral band being utilized by the system 10.

Referring again to FIG. 1, the multi-spectral imaging system 10 of the present invention may be utilized in connection with a mobile cart 15 for ease of use. The cart 15, in one embodiment, may include a positioning arm 152 to which the scanner 11 or 20 may be pivotally attached. The cart 15 may also include a stand 153 to which the arm 152 may be secured. The cart 15 may further include a housing 154 to which the stand 153 may be rigidly mounted. The housing 154, in an embodiment, may include multiple shelves (not shown) below surface 155, and on which, for instance, a power supply to the system 10, an external processor, and various other components and controller may be placed. As the system 10 may need to be moved for positioning over the object to be monitored and observed, the cart 15 may include wheels 156 to facilitate the relocation.

The dynamic imaging system 10 of the present invention may be adapted for a variety of uses. In one embodiment, the dynamic imaging system 10 can be used to obtain relatively fast and accurate images of the tissue area being evaluated and/or monitored. Moreover, since the imaging protocol employed by the system 10 can be based on the registration of passively emitted infrared photon flux from the tissue, the system 10 requires essentially no physical contact with the tissue or organ being evaluated and/or monitored.

Furthermore, the dynamic imaging system 10, can avoid using traditional contrast enhancement techniques, such as cooling the tissue area of interest or the use of contrast agents. By utilizing various algorithms which, among other things, can enhance the contrast between the vascular architecture and surrounding tissue, the system 10 can permit localization of the vascular architecture, while eliminating the need for cooling down the tissue area being observed (i.e., tissue area can be maintained at body temperature) and/or the need for using and delivering contrasting agents into the tissue.

In addition, the dynamic system 10 of the present invention can be used in a non-invasive manner to monitor, for instance, the physiological, functional, and/or structural characteristics of the tissue or organ being observed by analyzing the infrared energy that is emitted, absorbed or reflected from the tissue or organ. Specifically, a multi-spectral detector or multiple detectors may be used to collect, for example, infrared data from two or more bands and the intensity of each of the bands thereafter may be compared to determine tissue characteristics.

In particular, infrared photons, or “black body radiation”, emitted from the tissue and organ being monitored and observed may be collected as a stream of individual frames by the multi-spectral detector without the need to cool down the tissue. Subsequently, each frame may be analyzed and compared for changes in the photon flux. Changes in photon flux are typically the direct results of changes in tissue physiology. In one example, the multi-spectral system 10 may be used to identify/determine, among other things, an area of low blood flow in, for example, tissue graft, by comparing the intensity of the infrared flux at the absorption band of C0₂, about 3 to 5 μm, versus the absorption band of oxygenated hemoglobin, from about 0.1 to 2 μm, and preferably about 0.6 μm to 1.0 μm, or to any other band. This information could assist a surgeon in locating the optimal point for harvesting a graft.

Moreover, as there can be many biomedical applications which may require the ability to determine the concentration of naturally occurring or introduced chemicals in living tissue, the dynamic system 10 of the present invention may be used to measure, for instance, oxygen and/or C0₂ concentration to assist clinicians in assessing disease state and response to therapy. The ability to detect and measure, in vivo and in real time, concentrations of naturally occuring or introduced chemicals or gases in tissue or organs permit, in one embodiment, segmentations of data as a function of depth in tissue by comparing at least two frequencies that have different depth penetration capabilities, for example, about 3-5 μm vs. about 8-10 μm. The multi-spectral system 10 of the present invention, as should be noted, can be adapted to generate 3-D infrared data sets, which can further enhance this ability. In addition, detectors, including multi-spectral detectors, can be customized to track specific pharmacological substance introduced into tissue or be tuned to certain chemical by-products of disease, such as cancerous production of NO.

### Pre-Operative Evaluation

The dynamic imaging system 10 may also be used in pre-operative evaluation, for example, in the evaluation of the vascular architecture and more specifically, the perforator vessels. The ability to evaluate the vascular architecture can assist in the subsequent harvesting of a tissue flap, the assessment of the effects of diseases, such as cancer and diabetes, and the evaluation of vascular functional behavior using DAT, including non-infrared methods capable of monitoring changes in perfusion periodicity.

To perform a pre-operative evaluation of the vascular architecture prior to, for example, harvesting a tissue flap, in one embodiment, the objective portion on the scanner (e.g., scanner 11 or 20) of system 10 may be placed adjacent the area being evaluated, for instance, in front of a body part of a patient. It should be noted that the area being evaluated need not be cooled down and may be maintained at substantially normal body temperature when employing the system 10 of the present invention. Ruler 100, such as that shown in FIG. 10, may next be placed within the field of view of the scanner. A mark may thereafter be made on the body part using a surgical marker within the field of view of the scanner. In one embodiment, the mark may be made close to a center point of the ruler 100. The mark, in accordance with an embodiment of the present invention, can be used as a reference of a coordinate system.

Once the mark has been made, the dynamic imaging system 10 may be activated to permit the scanner to scan the area being evaluated. The scan, in one embodiment, may be about 20 seconds in length, but can vary in length of time, depending on the procedure. The system 10 may then process the data captured from the scan using, for example, one or a combination of the following algorithms: Spot FFT (Fast Fourier Transformation), Spot Standard Deviation, Spot Average.

The algorithms being employed with the system 10 may be applied to a collection of pixels called spots. In an example, a spot of size 1 represents one pixel, whereas a spot of size 2 represents collection of four adjacent pixels, and a spot of size 3 represents an area of 3×3 pixels—a collection of nine adjacent pixels. The average value of intensity of thermal photon flux may be calculated for the spot.

In connection with the evaluation protocol employed by the dynamic imaging system 10 of the present invention, Spot Average may be used to localize spots of increased temperature specific to the locations of perforator vessels. Spot Average includes the calculation of the average temperature value for the pixels included in the spot. Spot average temperature, once calculated, may be displayed in a form of a data matrix (frame). The values of the spot average temperature may be displayed in a gray scale mode or may be color coded according to a pseudo color palette. Spot average temperature matrices represent temperature distribution over the field of view. Spot average temperature distribution may represent temperature distribution in a single frame selected or summarized spot average temperature distribution for the collection of frames. The values of the spot average temperature may be displayed in a form of a graph representing spot average temperature values as a function of time of the scan.

Spot Standard Deviation, on the other hand, may be used to localize spots of increased temperature variation specific to the location of perforator vessels. Spot Standard Deviation includes calculation of the value of the standard deviation for the pixels included in the spot over the duration of the scan. Spot standard deviation represents the variation of the temperature distribution with reference to the average value of the temperature calculated for the time series of the duration of the scan. This algorithm represents the magnitude of temperature variations within a field of view during the scan. The values of spot standard deviation may be displayed in a form of a data matrix (frame). The values of spot standard deviation may be displayed in a gray scale mode or may be color coded according to the pseudo color palette. Spot standard deviation matrices may represent distribution of the magnitude of temperature variation with reference to the average temperature over the field of view. Spot standard deviation distribution may represent standard deviation distribution in a single frame selected or summarized spot standard deviation distribution for the collection of frames. The values of the spot standard deviation may be displayed in a form of a graph representing spot standard deviation values as a function of time of the scan.

Spot FFT may be used to localize spots of increased temperature modulation specific to the location of perforator vessels. Spot FFT calculates the frequency power spectrum distribution in the time series for each spot in the frames and the time series for each spot over the duration of the scan may be processed. Spot FFT represents the distribution of intensity of temperature modulation for the selected frequency ranges. Spot FFT temperature modulation intensities may be then displayed in a form of data matrix (frame). The values of spot FFT temperature modulation may be displayed in a gray scale mode or may be color coded according to the pseudo color palette. Spot FFT temperature modulation matrices may represent distribution of the magnitude of temperature modulation within selected frequency range over the field of view. Spot FFT temperature modulation distribution may represent temperature modulation distribution of a summarized spot FFT temperature modulation distribution for the collection of frames.

Combinations of indications by the above algorithms, in an embodiment, can be used to determine the location of perforator vessels on the tissue surface of examined area.

Once the scanned data have been processed, the results of the processed data may thereafter be presented as an image or images on the display screen of the imaging system 10. The image or images presented on the display screen, in one embodiment, may be pseudo-color or color images for evaluation. It should be appreciated that the image or images on the display screen may initially present areas of high power distribution at the locations of the perforators. Subsequently, for each algorithm employed, an operator may, if desired, manipulate the imaging system 10, including for example, selecting and narrowing different parameters, such as frequency and/or temperature range, to further enhance the contrast between, for instance, the perforator vessels (i.e., those vessels comprising the vascular architecture) and surrounding tissue to show the distribution and location of perforator vessels. The resulting processed image can be presented on the display screen, for instance, with areas of high contrast showing the distribution and locations of perforator vessels.

Thereafter, calibration of the image may be performed for translation purposes, i.e., positioning and aligning the image onto the corresponding location on the body of the patient. Specifically, the operator may perform software assisted dimensional calibration of the image either manually or automatically. To calibrate the image manually, the Dimensional Calibration Application, for example, may be initiated. In particular, the calibration marks on the ruler 100 may be identified and a line may be drawn between the calibration marks, which line serves to calibrate the image. To determine locations of perforator vessels within an image frame, a Grid Application may be employed, whereby a grid may be overlaid on the captured image on the display screen. The location of the origin of the grid (i.e., the origin of the coordinate system for the image frame) may be modified, including rotating the grid, if necessary, to subsequently align it on the patient's body.

Next, the origin of the grid may be moved to the mark on the patient's body and, in an example, the grid may be adjusted to align it with an edge of the ruler 100. The Measurement Application may then be initiated. In particular, a line from the origin of the grid may be generated toward the location of the perforators. The distances measured on the image from the lines, in one embodiment, may be the same as the distances on the patient body. Once the measurements have taken place, the measurements may be transferred from the image to the body of the patient, and the locations of the perforator vessels may be marked thereon.

Alternatively, automated calibration may be employed using feature identification software to avoid manual selection. It should be appreciated that the localization of the perforator vessels, in a preferred embodiment, may be carried out by the imaging system 10 with a resolution of approximately ±2 mm.

The dynamic imaging system 10 of the present invention may further be used pre-operatively, for instance, to stage the advancement of a disease and its effect on perfusion, such as in the case of diabetic neuropathy, or to plan a surgical procedure in response to the pre-operative result by identifying the best method and location for intervention. The imaging system 10 may also be used to evaluate perfusion in organs to be used during the transplantation, and may have applications in monitoring changes in perfusion related to patient behavior, such as exercise and diet, for instance, in the case of diabetic neuropathy. In such a situation, the imaging system 10 may provide important screening or diagnostic information, so as to identify the existence, stage the advancement, or monitor the effects of behavioral modifications, chemotherapy, surgical intervention or other medical or physical therapy over the lifetime of the patient.

### Post-Operative Evaluation

The imaging system 10 may also be used in connection with post-operative evaluation, for instance, blood perfusion within a tissue graft or transplanted organ.

To perform a post-operative evaluation of, for example, a tissue flap subsequent to the transplantation, in one embodiment, the objective portion on the scanner (e.g., scanner 11 or 20) of system 10 may be placed adjacent the tissue graft so that blood perfusion during the post-operative period may be evaluated. As with the pre-operative evaluation, the area being evaluated need not be cooled down and may be maintained at substantially normal body temperature. Thereafter, operator may set parameters, for example, parameters similar to that in a Sequential Scanning Protocol. This protocol, in one embodiment, allows the taking of a series of images over an extended period of time (seconds, minutes, hours, days etc.). The number of image frames, the total period of image collection, and the pause between consecutive collections can be set. The analysis of the collected images allows instant evaluation of the blood perfusion during the postoperative period.

In the same way, and for similar reasons, the dynamic imaging system 10 of the present invention may be useful for post-operative monitoring of other surgical procedures including, but not limited to, rejoining of limbs following traumatic amputation, transplantation of organs or tissue, and interventional vascular procedures, such as angioplasty and stenting of vessels.

It should be noted that the design of the dynamic imaging system 10 and the methods of operating same provides ease of use and makes the system easily adaptable to various applications. For instance, the processor used in connection with the system 10 may be easily adapted for the particular application in use. Specifically, the type of application to be implemented can determine the type of algorithm that is to be used. For instance, whether the intention is to analyze massive changes of blood flow in major blood vessels, or minute changes in capillaries or cellular metabolic behavior, the system 10 can be easily adjusted and optimized by the user through an easy-to-use interface. Moreover, the configuration of the system 10 can lend itself to being used, for instance, in either a conventional wide-field surgery or alternately during minimally invasive surgery through the use of an endoscopic accessory lens. The design of system 10, depending on the tissue area under investigation can also allow the present system to be used outside of a sterile field.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims. It should be noted that although surgical procedures are suggested, the potential uses for system 10 is not limited to such, or for that matter, medical applications.

What is claimed is:

1. A dynamic imaging system comprising:
a scanner having a body portion and an objective portion;
an assembly, positioned within the objective portion, for splitting a photon beam emitted from a tissue area being monitored into multiple incident rays of different wavelength spectra;
a detection network, designed to receive the multiple incident rays, for converting, into electronic signals, data correlated from the incident rays;
a processor in communication with the detector for generating discrete image data from the electronic signals of each respective incident ray for subsequent display as an image;
a display for viewing the image data; and
at least one ruler for positioning on the tissue area being monitored to permit subsequent translation of the image viewed in the display onto the tissue area.

2. An imaging system as set forth in claim 1, wherein the detection network includes multiple detectors, each being tuned to a specific wavelength spectrum of the incident ray it is collecting.

3. An imaging system as set forth in claim 2, wherein the detectors are single-band detectors.

4. An imaging system as set forth in claim 2, wherein at least one of the detectors is tuned to detect photons within an infrared spectrum.

5. An imaging system as set forth in claim 4, wherein the detector tuned to detect photons within an infrared spectrum is a quantum well infrared photodetector.

6. An imaging system as set forth in claim 2, wherein at least one of the detectors is a multi-spectral detector.

7. An imaging system as set forth in claim 1, wherein the detection network includes a multi-spectral detector.

8. An imaging system as set forth in claim 1, wherein the detection network includes a single-band detector.

9. An imaging system as set forth in claim 1, further including a lens system through which photon beams within the infrared spectrum emitted from an object being monitored may be directed for providing an image of the tissue area.

10. An imaging system as set forth in claim 9, wherein the lens system includes multiple lenses for providing binocular or three dimensional images of the tissue area.

11. An imaging system as set forth in claim 1, wherein the ruler includes marks at predetermined calibrated distances for visualization in the field of view of the scanner.

12. A dynamic imaging system comprising:
an objective portion through which a photon beam emitted from a tissue area being monitored may be directed;
a plurality of mirrors, positioned within the objective portion, for splitting the photon beam into multiple incident rays, each within a different wavelength spectrum;
at least one detector, the detector being tuned to a specific wavelength spectrum of the incident ray it is collecting from the corresponding mirror, so as to subsequently convert, into electronic signals, data correlated from the respective incident ray;
a processor for generating discrete image data from the electronic signals of each respective incident ray for subsequent display as an image;
a display for viewing the image data; and
at least one ruler for positioning on the tissue area being monitored to permit subsequent translation of the image viewed in the display onto the tissue area.

13. A method for evaluating vascular architecture of a tissue area on a patient, the method comprising:
maintaining, at substantially normal body temperature, the tissue area having the vascular architecture of interest on the patient;
detecting photon flux emitted from the tissue area;
processing data collected from the detected photon flux;
enhancing contrast between the vascular architecture and surrounding tissue within the area being scanned; and
generating an image from the processed data for display.

14. A method as set forth in claim 13, wherein the step of detecting includes collecting a stream of individual frames of data from the detected photon flux.

15. A method as set forth in claim 13, further including evaluating, at a pre-operative period, the displayed image.

16. A method as set forth in claim 15, wherein the step of evaluating includes determining blood flow in the vascular architecture.

17. A method as set forth in claim 15, wherein the step of evaluating includes determining perfusion within the vascular architecture for subsequent harvesting of the tissue area.

18. A method as set forth in claim 15, wherein the step of evaluating includes identifying existence of a condition in the patient based on perfusion within the vascular architecture.

19. A method as set forth in claim 15, wherein the step of evaluating includes assessing advancement of a condition in the patient based on perfusion within the vascular architecture.

20. A method as set forth in claim 15, wherein the step of evaluating includes monitoring effects of patient behavioral modification based on perfusion within the vascular architecture.

21. A method as set forth in claim 13, further including evaluating, at a post-operative period, the displayed image.

22. A method as set forth in claim 21, wherein prior to evaluating, the step of scanning includes taking a series of sequential images over an extended period of time of the tissue area being evaluated.

23. A method as set forth in claim 21, wherein the step of evaluating includes monitoring perfusion within the vascular architecture of the post-operative tissue area to determine the health of the tissue area.

24. A method as set forth in claim 23, wherein in the step of monitoring the health of the tissue area is determined in connection with one of a rejoining of limb, a transplantation, and a interventional vascular procedure.

25. A method localizing perforator vessels, the method comprising:
maintaining, at substantially normal body temperature, a tissue area within which the perforator vessels are located;
placing a reference point on the tissue area to assist in subsequent localization;
scanning the tissue area with an infrared camera, so that the reference point is within the field of scan, to detect photon flux emitted therefrom;
processing data collected from the detected photon flux;
enhancing contrast between the perforator vessels and surrounding tissue within the area being scanned; and
generating, relative to the reference point, an image from the processed data for display.

26. A method as set forth in claim 25, wherein the step of placing includes providing a ruler having calibrated markings for use in connection with dimension calibration of the tissue area.

27. A method as set forth in claim 26, wherein the step of providing includes positioning the reference point substantially close to a center point of the ruler.

28. A method as set forth in claim 25, wherein the step of processing includes application of one of Spot Fast Fourier Transformation, Spot Standard Deviation, Spot Average, or a combination thereof to determine location of location of the perforator vessels in the tissue area.

29. A method as set forth in claim 25, wherein the step of enhancing includes selecting and narrowing frequency range, temperature range, or both to identify distribution and location of the perforator vessels.

30. A method as set forth in claim 25, further comprising:
positioning on the image displaying the perforator vessels an electronic illustration of a grid having a coordinate system, such that its origin is situated relative to the reference point captured during the scan;
identifying location of the perforator vessels within the grid;
while maintaining orientation of the grid relative to reference point, translating the location of the perforator vessels within the grid to the tissue area previously scanned; and
marking on the tissue area the location of the perforator vessels as identified within grid.

31. A method as set forth in claim 30, wherein the step of positioning includes maintaining alignment and orientation of the grid origin relative to the captured reference point.

32. A method as set forth in claim 30, wherein the step of identifying includes measuring within the grid a distance between a perforator vessel and the origin.
